(19) United States
(12) Patent Application Publication
(10) Pub. No.: US 2011/0270016 A1
(43) Pub. Date: Nov. 3, 2011

(54) SELF-ADJUSTING GASTRIC BAND

(75) Inventors: Sean Snow, Carpinteria, CA (US); Marcos Borrell, Goleta, CA (US)

(73) Assignee: ALLERGAN, INC., Irvine, CA (US)

(21) Appl. No.: 12/770,581

(22) Filed: Apr. 29, 2010

(51) Int. Cl. A61F 2/00

(52) U.S. Cl. 600/37

(57) ABSTRACT

A self-adjusting gastric band comprises an inflatable portion that includes a fluid such as saline. A reservoir is spaced from the inflatable portion according to a distance related to a wavelength of a peristaltic wave that propagates through the esophageal-gastric junction. A pressure-relief valve is coupled between the inflatable portion and the reservoir and allows an amount of the fluid to pass from the inflatable portion to the reservoir when a first force generates an increased pressure in the inflatable portion. The amount of fluid is released to allow the large bolus to pass through the esophageal-gastric junction. A contracted portion of the peristaltic wave is proximate the reservoir when an expanded portion of the wave is proximate the inflatable portion of the self-adjusting gastric band, to facilitate allowing the amount of the fluid to enter the reservoir.

### FIELD

The present invention generally relates to medical systems and apparatus and uses thereof for treating obesity and/or obesity-related diseases, and more specifically, relates to gastric banding systems that self-adjust to changes in a patient.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach may provide a feeling of satiety or fullness that discourages overeating. Unlike gastric bypass procedures, gastric band apparatus are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

Sometimes, adjustment of a gastric band may be desirable in between adjustments made by a physician. For example, during normal operation of the gastric band, the band applies pressure to the outer surface of the upper stomach. But in some instances, the patient may swallow a bolus that is too large to pass through the constriction produced by the band. The result can be a painful experience which, if it persists, may require medical intervention to release the blockage.

Some attempts have been made to account for this possibility of a blockage. For example, Steffen, U.S. Patent Pub. No. 2009/0062826 discloses a gastric band with a “conveyance device” that is powered by a “power storage device.” The power storage device operates the conveyance device to move fluid between expandable chambers to adjust the gastric band.

Coe, et al., U.S. Patent Pub. No. 2009/0216255 discloses a flow control device that moves fluid between a hydraulic restriction system and a fluid source. The additional flow control component controls a rate of fluid flow between the restriction device and the fluid source. Coe, et al., European Patent Application No. 2 074 970 A1 discloses a separate restriction device and pressure adjustment device. The pressure adjustment device regulates a constant force applied by the restriction device.

Lechner, U.S. Patent Pub. No. 2009/0054914 discloses a controllable stomach band that has a chamber for controlling restriction of the stomach band. The chamber is coupled to a separate pressure chamber that receives fluid leaving the chamber in the stomach band. The chamber is separated from the esophageal-gastric junction.

Accordingly, it is desirable to develop a self-adjusting gastric band that will provide the needed pressure to the stomach to create the stoma and facilitate weight control, but that will also adjust and open up to allow a large bolus to pass through. It is further desirable to create an automatically self-adjusting gastric band that does not require an electrical power source and/or external adjustments, to allow a large bolus to pass through.

Additionally, it is desirable to make the adjustments without additional, complicated fluid control mechanisms to regulate the transfer of fluid within the self-adjusting gastric band. Moreover, it is desirable to make these adjustments to the gastric band utilizing peristaltic waves to both reduce and restore the constriction of the gastric band.

### SUMMARY

Generally described herein are self-adjusting gastric banding systems that automatically expand and contract in response to a large bolus passing through the area of a patient's stomach constricted by a gastric band. The apparatus and systems described herein aid in facilitating obesity control and/or treating obesity-related diseases while being non-invasive once implanted.

In one embodiment, a self-adjusting gastric band is disposable about an esophageal-gastric junction of a patient. The self-adjusting gastric band comprises a rigid ring and an inflatable portion on the inside of the rigid ring. The inflatable portion includes a fluid such as saline.

The self-adjusting gastric band also comprises a reservoir that is spaced from the inflatable portion according to a distance related to a wavelength of a peristaltic wave that propagates through the esophageal-gastric junction. For example, the distance may be approximately equal to ⅛, ¼, ½, or 1 wavelength, or other wavelength that facilitates automatic self-adjustment of the gastric band. The peristaltic wave includes an expanded portion that exerts a first force on the inflatable portion when a large bolus passes through the esophageal-gastric junction.

In an embodiment, a pressure-relief valve is coupled between the inflatable portion and the reservoir. The pressure-relief valve allows an amount of the fluid to pass from the inflatable portion to the reservoir when the first force generates an increased pressure in the inflatable portion. The amount of fluid is released to allow the large bolus to pass through the esophageal-gastric junction.

Further, in accordance with an embodiment, the peristaltic wave comprises a contracted portion proximate to the expanded portion of the peristaltic wave. The expanded portion is near a peak of the peristaltic wave and the contracted portion is near a trough of the peristaltic wave. The contracted portion of the peristaltic wave is proximate to the reservoir when the expanded portion is proximate to the inflatable portion of the self-adjusting gastric band, to facilitate allowing the amount of the fluid to enter the reservoir.

In another embodiment, the self-adjusting gastric band comprises a one-way valve coupled between the reservoir and the inflatable portion of the gastric band. The amount of the fluid flows through the one-way valve from the reservoir to the inflatable portion after the large bolus passes through the esophageal-gastric junction. Further, the expanded portion of the peristaltic wave is proximate the reservoir when the inflatable portion is proximate to the contracted portion of the peristaltic wave to facilitate moving the fluid from the reservoir to the inflatable portion of the self-adjusting gastric band.

In various embodiments, the self-adjusting gastric band is coupled to an access port for filling and draining the inflatable portion of the gastric band. The pressure-relief valve and/or the one-way valve may be proximate to the inflatable portion, the reservoir, and/or the access port. The pressure-relief valve and/or the one-way valve may be adjustable prior to and/or after implantation of the self-adjusting gastric band in the patient.

Additionally, in accordance with an embodiment, the self-adjusting gastric band comprises two pressure relief valves. The first pressure-relief valve is set to a first burst pressure corresponding to a maximum pressure for the inflatable portion. The maximum pressure is reached in the inflatable portion when the large bolus moves through the esophageal-gastric junction. The second pressure-relief valve is set to a second burst pressure that allows the portion of the fluid to flow from the reservoir to the inflatable portion when a reservoir pressure is greater than a minimum inflatable portion pressure.

According to various embodiments, a method for automatically adjusting a gastric band permits a large bolus of food to pass through an esophageal-gastric junction of a patient. The method comprises implanting the self-adjusting gastric band around the patient's esophageal-gastric junction. The self-adjusting gastric band comprises an inflatable portion for holding a fluid and a reservoir coupled to the inflatable portion via a pressure-relief valve.

The method further comprises adjusting the pressure-relief valve in accordance with a predetermined pressure level of the inflatable portion, and causing a portion of the fluid to exit the inflatable portion through the pressure-relief valve. The fluid exits the inflatable portion when a pressure in the inflatable portion reaches the predetermined pressure level. The predetermined pressure level is reached when the large bolus passes through the esophageal-gastric junction.

Additionally, the method comprises relaxing a constriction created by the self-adjusting gastric band in response to the portion of the fluid exiting the inflatable portion. Relaxing the constriction permits the large bolus to pass through the esophageal-gastric junction. The portion of the fluid exits the reservoir through a one-way valve after the large bolus passes through the esophageal-gastric junction. Returning the fluid to the inflatable portion restores the constriction created by the self-adjusting gastric band.

### BRIEF DESCRIPTION OF THE DRAWINGS

FIG. 1A illustrates a perspective, cut-away view of a gastric band with an inflatable portion according to an embodiment of the present invention.

FIG. 1B illustrates a side, cross-sectional view of the embodiment of the self-adjusting gastric band illustrated in FIG. 1A.

FIG. 2A illustrates a schematic representation of a self-adjusting gastric banding system according to an embodiment of the present invention.

FIG. 2B illustrates a schematic representation of a self-adjusting gastric banding system with valves proximate an access port according to an embodiment of the present invention.

FIG. 3A illustrates a sectional view of a patient's stomach with a self-adjusting gastric band constricting an esophageal-gastric junction of the stomach according to an embodiment of the present invention.

FIG. 3B illustrates a large bolus of food passing through an esophageal-gastric junction of a patient's stomach, with a peristaltic wave exerting a force on an inflatable portion of a self-adjusting gastric band according to an embodiment of the present invention.

FIG. 3C illustrates a reduced constriction of a self-adjusting gastric band to allow a bolus to pass through the constriction according to an embodiment of the present invention.

FIG. 3D illustrates a force exerted by a peristaltic wave on a reservoir of a self-adjusting gastric band to facilitate returning a portion of a fluid to the inflatable portion of the self-adjusting gastric band according to an embodiment of the present invention.

### DETAILED DESCRIPTION

The present invention generally provides self-adjusting gastric banding systems, for example, for treatment of obesity and obesity related conditions, as well as systems for automatically controlling adjustment of gastric bands when a patient swallows a large bolus.

Adjustable gastric bands are effective in helping a patient lose weight when the band is properly tightened around the patient's esophageal-gastric junction. During normal operation, the band applies pressure to the outer surface of the upper stomach. But, in some instances, the patient may swallow a bolus which is too large to pass through the constriction produced by the band—for example, when the patient swallows a large piece of steak. The result can be a painful experience which, if it persists, may require medical intervention to release the blockage.

In accordance with various embodiments of the present invention, the self-adjusting gastric band provides the needed pressure to the stomach to encourage weight loss. However, when a large bolus of food is swallowed, the self-adjusting gastric band temporarily opens up to allow the bolus through. After the bolus passes through, the mechanisms within the band return the band to its original size and shape. In an embodiment, electrical power and/or power external to the patient is not utilized to perform these adjustments.

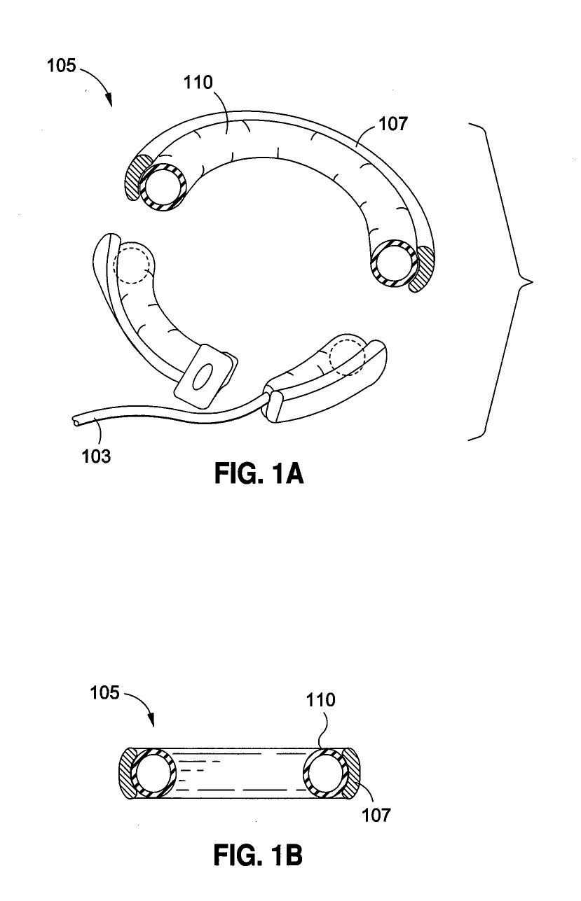

Turning now to FIGS. 1A-1B, a self-adjusting gastric band 105 comprises a circular, rigid ring 107 and an inflatable portion 110 disposed on the inside of the rigid ring 107. The inflatable portion 110 separates the patient's stomach from the rigid ring 107 when the gastric band 105 is implanted around the esophageal-gastric junction of the patient's stomach. The rigid ring 107 provides structure and support to the inflatable portion 110, and facilitates implanting the gastric band 105 around the patient's stomach.

The inflatable portion 110 may be filled and drained with a fluid via tubing 103. For example, the tubing 103 may be connected to a subcutaneous access port for filling and draining the inflatable portion 110 via subcutaneous injections. The inflatable portion 110 may also be coupled to a reservoir to facilitate automatic adjustment of the inflatable portion 110, and the constriction it causes, when a large bolus attempts to pass through the constriction. When more fluid is present in the inflatable portion 110, the constriction around the stomach becomes tighter. Correspondingly, when less fluid is present, the constriction loosens and/or opens up.

The fluid used within the gastric band 105 includes any fluid that is biocompatible and incompressible. The fluid has no adverse effect on the patient in the unlikely event that a leak emanates from the system. The fluid can simply be water, or any biocompatible polymer oil such as caster oil. In an example embodiment, the fluid is saline, a drug, and/or combinations thereof.

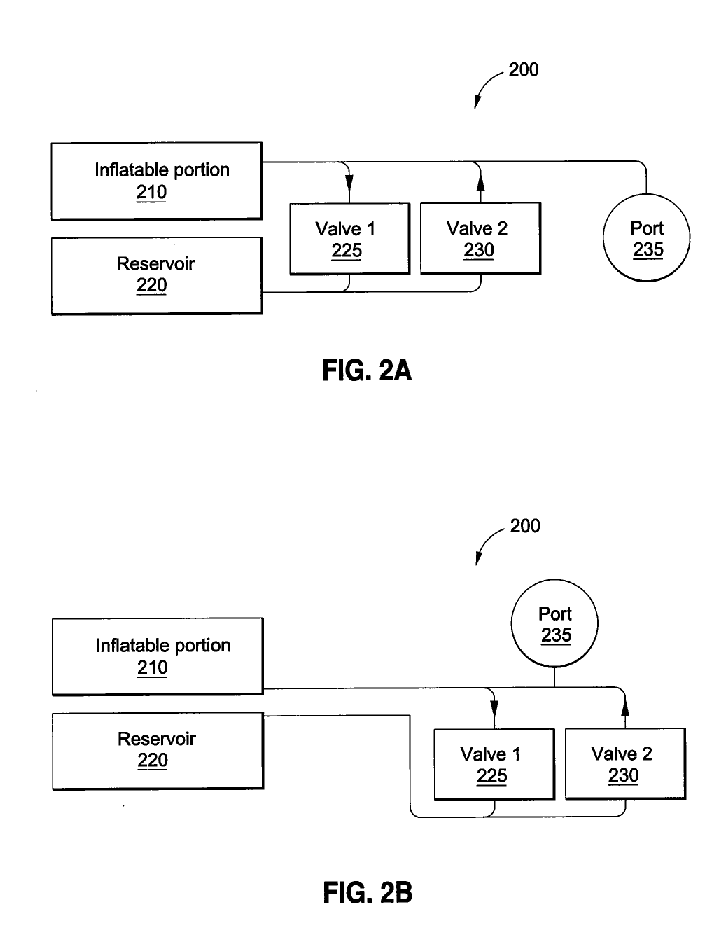

In various embodiments, and with reference to FIGS. 2A-2B, a gastric banding system 200 includes the inflatable portion 210 coupled to a reservoir 220 receiving fluid from and sending fluid to the inflatable portion 210. When a large bolus passes through the constriction in the stomach caused by the inflatable portion 210, the reservoir 220 may receive fluid from the inflatable portion 210 in order to relax the constriction in the stomach and to allow the bolus to pass through the constriction. When the bolus passes, the reservoir 220 may send fluid to the inflatable portion 210 in order to restore the constriction.

A first valve 225 may control the flow of the fluid from the inflatable portion 210 to the reservoir 220. Similarly, a second valve 230 may control the flow of the fluid from the reservoir 220 to the inflatable portion 210. In other embodiments, a single valve may be used to control flow into and out of the inflatable portion 210 and the reservoir 220.

In an embodiment, the first valve 225 is a pressure-relief valve with a burst pressure set to allow fluid to flow from the inflatable portion 210 to the reservoir 220 when a predetermined pressure level is reached within the inflatable portion 210. For example, when a large bolus passes through the constriction caused by the inflatable portion 210, the pressure in the inflatable portion 210 increases because the bolus exerts a force on the wall of the stomach near the inflatable portion 210. If this pressure increase rises above the predetermined pressure level, the burst pressure for the pressure-relief valve 225 is reached, and fluid flows from the inflatable portion 210 to the reservoir 220.

Further, in an embodiment, the second valve 230 is a one-way valve that only allows fluid to flow from the reservoir 220 to the inflatable portion 210. In this manner, when the pressure in the inflatable portion 210 returns below the predetermined pressure level, the fluid returns to the inflatable portion 210 to restore the desired amount of constriction to the patient's stomach.

In accordance with another embodiment, both valves 225, 230 may be pressure-relief valves. Each pressure-relief valve 225, 230 is set to a different burst pressure or opening pressure. For example, the valve 225 may be set to a burst pressure corresponding to a maximum pressure for the inflatable portion 210, such that if the pressure exceeds the maximum pressure, the valve 225 will open and allow fluid to flow into the reservoir 220. Then, if the pressure in the inflatable portion 210 drops below a minimum pressure, the valve 230 will open, causing fluid to flow from the reservoir 220 into the inflatable portion 210. In this manner, the pressure in the inflatable portion 210 is constrained in a specific range. When this specific range is properly adjusted, the specific pressure range may constrain the gastric band to a beneficial therapeutic range of constriction.

The inflatable portion 210 and the reservoir 220 may be fluidly coupled in any manner that allows fluid to flow between the inflatable portion 210 and the reservoir 220. The inflatable portion 210 may be coupled to the reservoir 220 via flexible tubing. In an embodiment, the inflatable portion 210 and the reservoir 220 may be similarly-shaped, elongated balloons (as illustrated for the inflatable portion 110 in FIG. 1A). These elongated balloons may be connected along their length with the valves 225, 230 coupled between the elongated balloons.

The inflatable portion 210 and/or the reservoir 220 may comprise a compressible reservoir, such as an elastic polymer, a balloon, a rubber container, a silicone container, a collapsible container, a non-elastomeric container, a bellows, and combinations thereof that are configured to contain the fluid. The inflatable portion 210 and/or the reservoir 220 may be the same shape, material, and/or durometer, or they may have different physical or chemical properties or parameters.

The gastric banding system 200 further comprises an access port 235 for filling and draining the inflatable portion 210 of the gastric band. The access port 235 may be sutured onto the rectus muscle sheath or any other conveniently accessible muscle. The rectus muscle sheath provides a secure surface on which to attach the access port 235 under a layer of fat that separates the patient's skin from the muscle. As illustrated in FIG. 2A, in an embodiment, the valves 225, 230 are coupled near the inflatable portion 210 and the reservoir 220. In an embodiment as illustrated in FIG. 2B, the valves 225, 230 are coupled near the access port 235. When the valves 225, 230 are coupled near the access port 235, the valves 225, 230 may be adjustable (e.g., to adjust the burst pressure(s)) after implantation using a hypodermic needle, a hypodermic tool, a magnetic field, and the like. Further, the valves 225, 230 may be adjustable by an additional surgery by reopening the site of the port 235.

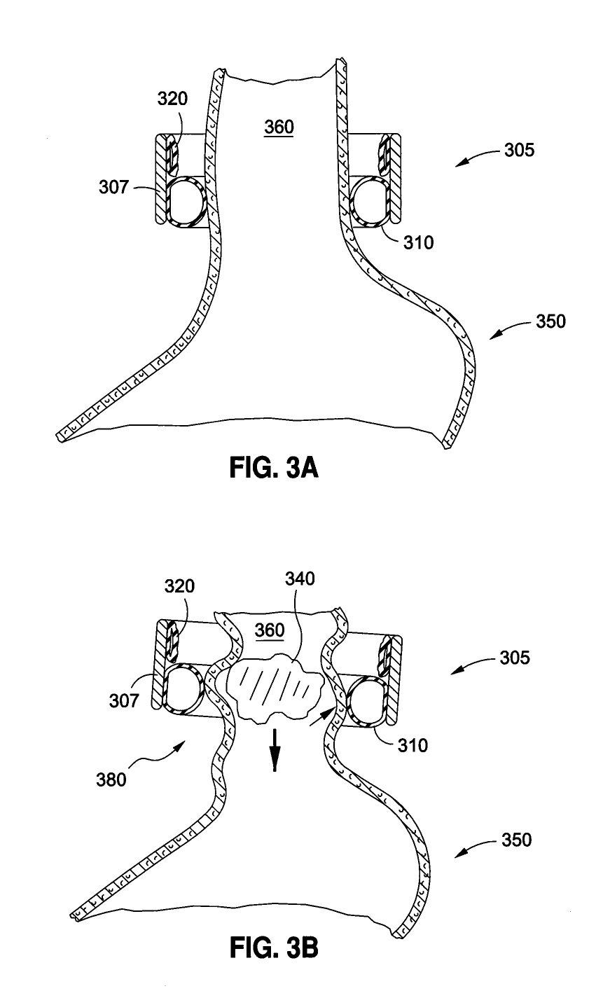

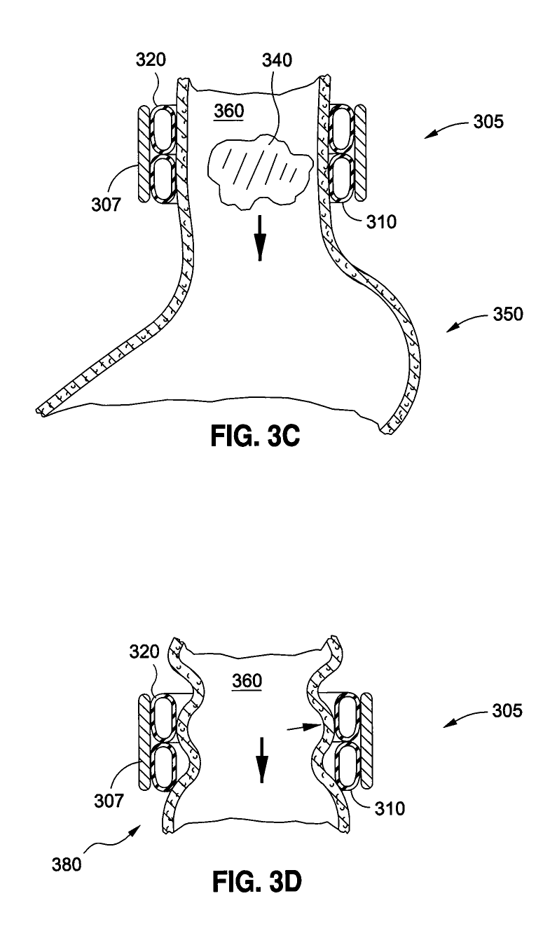

Turning to FIGS. 3A-3D, operation of the self-adjusting gastric band 305, according to an embodiment, will now be disclosed. After the gastric band 305 has been implanted around an esophageal-gastric junction 360 of the patient's stomach 350, the gastric band 305 constricts the esophageal-gastric junction 360 to facilitate weight loss. The inflatable portion 310 contains a fluid, while the reservoir 320 is substantially empty.

As illustrated in FIG. 3A, a single ring 307 may provide structure and/or support to both the inflatable portion 310 and the reservoir 320. However, it should be understood that a separate ring may be used for each of the inflatable portion 310 and the reservoir 320. And these separate rings may be directly next to each other, or they may be advantageously separated and/or separated by a predetermined distance, for example, they may be separated by a distance related to a wavelength of a peristaltic wave, as discussed further below. In various embodiments, the inflatable portion 310 may be separated from the reservoir 320 by a distance between approximately zero to three inches.

As the patient swallows a large bolus 340 of food, and with reference to FIG. 3B, the peristaltic waves 380 force the bolus 340 into the esophageal-gastric junction 360. An expanded portion of the esophageal-gastric junction 360, caused by the bolus 340 and the peristaltic waves 380, increases the pressure in the inflatable portion 310 by exerting a force (designated by the small arrow in FIG. 3B) on the walls of the stomach 350 near the esophageal-gastric junction 360. In various embodiments, normal pressures for the inflatable portion 310 are approximately zero to fifteen pounds per square inch.

The inflatable portion 310 and the reservoir 320 are advantageously positioned so that at least a portion of a peak of the peristaltic wave 380 contacts the inflatable portion 310 while at least portion of a valley of the peristaltic wave 380 passes by the reservoir 320. The separation distance and/or size of the inflatable portion 310 and the reservoir 320 are thus determined in accordance with an expected wavelength of the peristaltic wave 380. For example, the separation distance and/or size of the inflatable portion 310 may be ⅛, ¼, ½, 1, or greater wavelength of the peristaltic wave 380 that allows the gastric band 305 to self-adjust.

The increased pressure in the inflatable portion 310 caused by the bolus 340 and the expanded portion of the esophageal-gastric junction 360 generated by the peak of the peristaltic wave 380, causes the pressure to increase past a maximum pressure, triggering the pressure relief valve. In an embodiment, the maximum pressure that triggers the pressure-relief valve is approximately eighteen pounds per square inch. When the maximum pressure in the inflatable portion 310 is reached, the pressure relief valve allows some of the fluid in the inflatable portion 310 to flow into the reservoir 320.

This reduction in fluid, as illustrated in FIG. 3C, makes the stoma of the inflatable portion 310 larger, so that the bolus 340 can pass through the gastric band 305. This configuration of the gastric band 305 is referred to as a “released” condition, because the constriction of the gastric band 305 has been released to allow the bolus 340 to pass through.

It should be understood from the above description, that the configuration of the gastric band 305 is advantageously determined to automatically adjust to the large bolus 340, such that no electrical and/or external power is utilized to redistribute the liquid from the inflatable portion 310 to the reservoir 320. The gastric band 305 is automatically adjustable, and uses the peristaltic wave 380 and the pressure created by the bolus 340 to redistribute the fluid and to release the constriction caused by the inflatable portion 310.

With reference also to FIG. 3D, and in accordance with an embodiment, once the large bolus 340 has passed through the gastric band 305, the peristaltic waves 380 facilitate returning the liquid from the reservoir 320 to the inflatable portion 310 to restore the proper constriction of and pressure in the inflatable portion 310.

The elastic nature of the reservoir 320, in conjunction with a peak of the peristaltic wave 380 that passes the reservoir 320 at substantially the same time as a valley of the wave passes the inflatable portion 310, causes the fluid in the reservoir 320 to flow through the one-way valve into the inflatable portion 310. The peak of the wave causes an increased pressure in the reservoir 320, by exerting a force (represented by the small arrow in FIG. 3D) on the wall of the stomach 350, that helps restore the fluid to the inflatable portion 310.

This process of restoring the fluid may be referred to as a “resetting” mode of the gastric band 305. Once the resetting mode has been completed, the gastric band 305 is again in the normal configuration, with the inflatable portion 310 constricting the esophageal-gastric junction 360 to the proper degree. Thus, the gastric band 305 is fully and automatically self-adjustable, for example, to expand and contract when a large bolus passes through the band. This automatic adjustment overcomes difficulties associated with prior art gastric bands that may have required physician intervention in the case where a patient swallowed too large of a bolus of food.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A self-adjusting gastric band disposable about an esophageal-gastric junction of a patient, the self-adjusting gastric band comprising:
a rigid ring;
an inflatable portion on the inside of the rigid ring, the inflatable portion including a fluid;
a reservoir spaced from the inflatable portion according to a distance related to a wavelength of a peristaltic wave propagating through the esophageal-gastric junction, an expanded portion of the peristaltic wave exerting a first force on the inflatable portion when a large bolus passes through the esophageal-gastric junction; and
a pressure-relief valve coupled between the inflatable portion and the reservoir, the pressure-relief valve allowing an amount of the fluid to pass from the inflatable portion to the reservoir when the first force generates an increased pressure in the inflatable portion, to allow the large bolus to pass through the esophageal-gastric junction.

2. The self-adjusting gastric band of claim 1 wherein the reservoir is on the inside of the rigid ring.

3. The self-adjusting gastric band of claim 1 further comprising a second rigid ring, the reservoir disposed on the inside of the second rigid ring.

4. The self-adjusting gastric band of claim 1 wherein the peristaltic wave propagates in response to the patient swallowing the large bolus of food.

5. The self-adjusting gastric band of claim 1 wherein the peristaltic wave further comprises a contracted portion proximate the expanded portion of the peristaltic wave.

6. The self-adjusting gastric band of claim 5 wherein the expanded portion is near a peak of the peristaltic wave and the contracted portion is near a trough of the peristaltic wave.

7. The self-adjusting gastric band of claim 5 wherein the contracted portion of the peristaltic wave is proximate the reservoir when the expanded portion is proximate the inflatable portion, to facilitate allowing the amount of the fluid to enter the reservoir.

8. The self-adjusting gastric band of claim 5 further comprising a one-way valve coupled between the reservoir and the inflatable portion of the gastric band.

9. The self-adjusting gastric band of claim 8 wherein the amount of the fluid flows through the one way valve from the reservoir to the inflatable portion after the large bolus passes through the esophageal-gastric junction, and when the expanded portion of the peristaltic wave is proximate the reservoir and when the inflatable portion is proximate the contracted portion of the peristaltic wave.

10. The self-adjusting gastric band of claim 8 further comprising an access port for filling and draining the inflatable portion of the gastric band.

11. The self-adjusting gastric band of claim 10 wherein at least one of the pressure-relief valve and the one-way valve is proximate at least one of the inflatable portion, the reservoir, and the access port.

12. The self-adjusting gastric band of claim 1 wherein the pressure-relief valve is adjustable prior to and after implantation of the self-adjusting gastric band in the patient.

13. The self-adjusting gastric band of claim 5 further comprising a second pressure-relief valve coupled between the reservoir and the inflatable portion of the gastric band.

14. The self-adjusting gastric band of claim 13 wherein the pressure-relief valve is set to a first burst pressure corresponding to a maximum pressure for the inflatable portion, and wherein the maximum pressure is reached in the inflatable portion when the large bolus moves through the esophageal-gastric junction.

15. The self-adjusting gastric band of claim 14 wherein the second pressure-relief valve is set to a second burst pressure that allows the portion of the fluid to flow from the reservoir to the inflatable portion when a reservoir pressure is greater than a minimum inflatable portion pressure.

16. The self-adjusting gastric band of claim 1 wherein the reservoir is spaced from the inflatable portion by a one-half wavelength of the peristaltic wave.

17. A method for automatically adjusting a self-adjusting gastric band to permit a large bolus to pass through an esophageal-gastric junction of a patient, the method comprising:
implanting the self-adjusting gastric band around the patient's esophageal-gastric junction, the self-adjusting gastric band comprising an inflatable portion for holding a fluid and a reservoir coupled to the inflatable portion via a pressure-relief valve;
adjusting the pressure-relief valve in accordance with a predetermined pressure level of the inflatable portion;
causing a portion of the fluid to exit the inflatable portion through the pressure-relief valve when a pressure in the inflatable portion reaches the predetermined pressure level in response to the large bolus passing through the esophageal-gastric junction; and
relaxing a constriction created by the self-adjusting gastric band in response to the portion of the fluid exiting the inflatable portion, to permit the large bolus to pass through the esophageal-gastric junction.

18. The method of claim 17 further comprising receiving the portion of the fluid into the reservoir, wherein the reservoir is substantially empty prior to receiving the portion of the fluid.

19. The method of claim 17 further comprising causing the portion of the fluid to exit the reservoir through a one-way valve after the large bolus passes through the esophageal-gastric junction.

20. The method of claim 19 further comprising restoring the constriction created by the self-adjusting gastric band when the portion of the fluid exits the reservoir and enters the inflatable portion of the self-adjusting gastric band.

21. The method of claim 16 wherein adjusting the pressure-relief valve comprises adjusting the pressure-relief valve by at least one of a hypodermic needle, a hypodermic tool, a magnetic field, and an additional surgery.

22. A self-adjusting gastric banding system that automatically adjusts a gastric band when a large bolus of food is swallowed by a patient, to allow the large bolus to pass through a constriction in the patient's stomach formed by the gastric band, the system comprising:
an inflatable portion of the self-adjusting gastric band, the self-adjusting gastric band being disposable about an esophageal-gastric junction of the patient;
a reservoir fluidly coupled to the inflatable portion of the self-adjusting gastric band, the reservoir separated from the inflatable portion in accordance with a distance related to a wavelength of a peristaltic wave formed when the patient swallows the large bolus, the peristaltic wave forming an expanded portion and a contracted portion of the esophageal-gastric junction, the expanded portion exerting a first force on the inflatable portion when the reservoir is proximate the contracted portion;
a pressure relief valve coupled between the inflatable portion and the reservoir, the pressure relief valve allowing an amount of fluid to move from the inflatable portion to the reservoir when the peristaltic wave moves through the gastric band, the first force exerted by the expanded portion of the esophageal-gastric junction on the inflatable portion causing the amount of fluid to move from the inflatable portion to the reservoir to automatically adjust the gastric band and to allow the large bolus to pass through the constriction in the patient's stomach;
a one-way valve coupled between the reservoir and the inflatable portion, the one-way valve allowing the amount of fluid to flow from the reservoir to the inflatable portion when the large bolus has passed through the constriction of the patient's stomach, the expanded portion of the peristaltic wave exerting a second force on the reservoir when the contracted portion is proximate the inflatable portion; and
an access port fluidly coupled to the inflatable portion of the self-adjusting gastric band for filling and draining the inflatable portion.
